(19) United States
(12) Patent Application Publication
(10) Pub. No.: US 2002/0107496 A1
(43) Pub. Date: Aug. 8, 2002

(54) Hygienic pad

(76) Inventor: Ya-Ching Wang, San-Chong City (TW)

Correspondence Address: CHARLES E. BAXLEY, ESQUIRE, Hart, Baxley, Daniels & Holton, 59 John St, 5 FI,, New York, NY, 10038, US

(21) Appl. No.: 09/776,945

(22) Filed: Feb. 6, 2001

(51) Int. Cl. A61F 13/15; A61F 13/20

(52) U.S. Cl. 604/385.01

(57) ABSTRACT

A hygienic pad includes a flat body having absorbing material received therein and a protrusion extends from a top surface of the body. The protrusion has a tapered ridge and is located to be inserted in the posterior groove of the user at the rear portion of the buttocks.

### FIELD OF THE INVENTION

The present invention generally relates to a hygienic pad having a protrusion from a top surface thereof and the protrusion is located in the posterior groove below the dimple so as to prevent the menses liquid from releasing from the buttocks.

### BACKGROUND OF THE INVENTION

FIG. 1 shows a conventional hygienic pad 101 that basically is an elongated absorbable pad so as to absorb the menses liquid. However, the length of the hygienic pad is not long enough to cover the rear part of the user's buttocks so that the menses liquid could flows from the posterior groove if the user lies down. FIG. 2 shows an improved hygienic pad 102 that has an enlarged extension portion which covers the rear part of the user's buttocks. Nevertheless, when the user lies down, a gap is formed between the hygienic pad and the user's posterior groove so that menses liquid still flows out from the gap. This becomes a problem especially when the user lies down, the gap is located at a lower position and the menses liquid has a nature tendency to flow toward the gap.

### SUMMARY OF THE INVENTION

The present invention relates to a hygienic pad that has a prism-shaped protrusion extending from a top surface of the body of the pad wherein the protrusion is inserted in the posterior groove of the user's buttocks so as to prevent menses liquid from flowing from the rear portion of the buttocks.

The primary object of the present invention is to provide a hygienic pad that effectively absorbs the menses liquid flowing from the posterior groove in a rear portion of buttocks.

These and other features, objects and advantages of the present invention will become more obvious from a careful reading of a detailed description provided herein below with appropriate reference to the accompanying drawings.

### BRIEF DESCRIPTION OF THE DRAWINGS

FIG. 1 is a perspective view to show a conventional hygienic pad;

FIG. 2 is a perspective view to show another embodiment of the conventional hygienic pad;

FIG. 3 a perspective view to show a hygienic pad of the present invention;

FIG. 4 is an illustrative view to show how the protrusion on the hygienic pad of the present invention to be inserted in the groove of the user's buttocks, and FIG. 5 is an illustrative view to show the position of the protrusion of the hygienic pad when the user lies down.

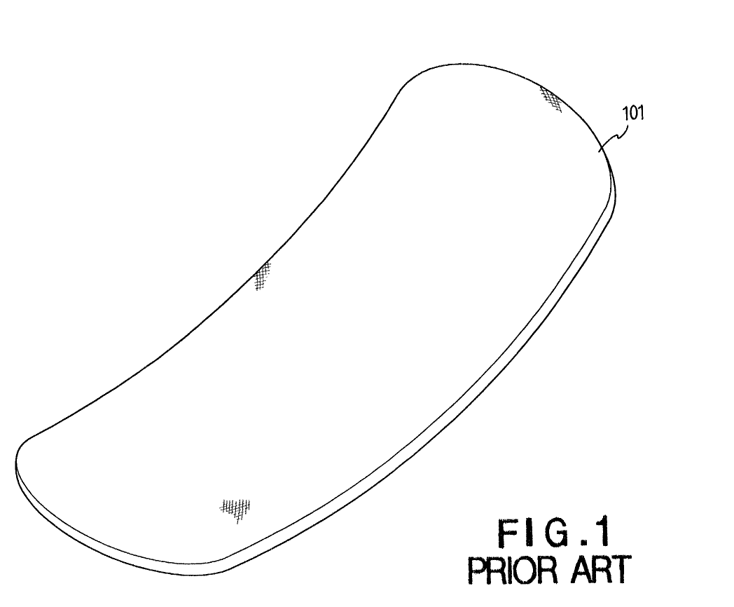

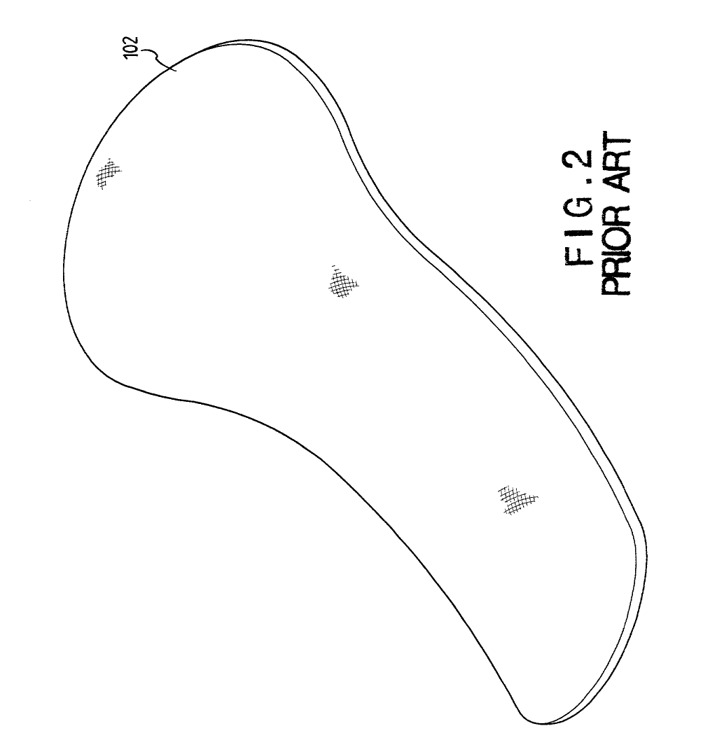

### DETAILED DESCRIPTION OF THE INVENTION

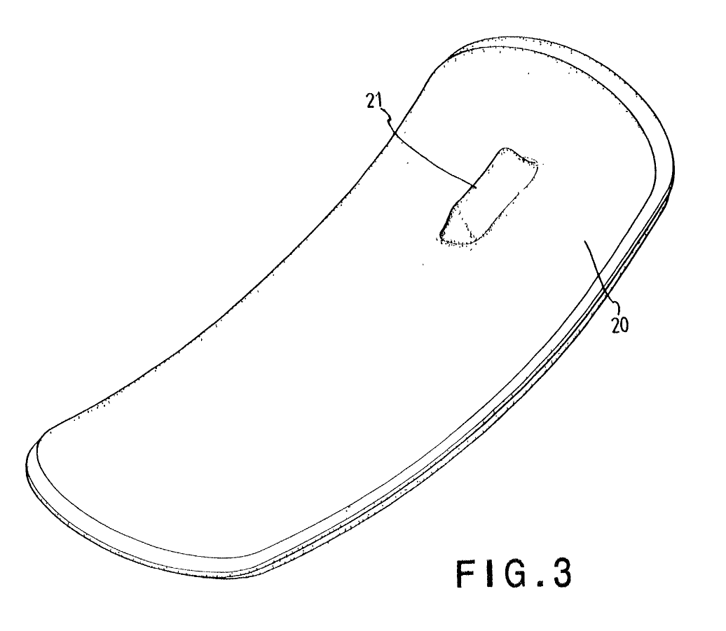

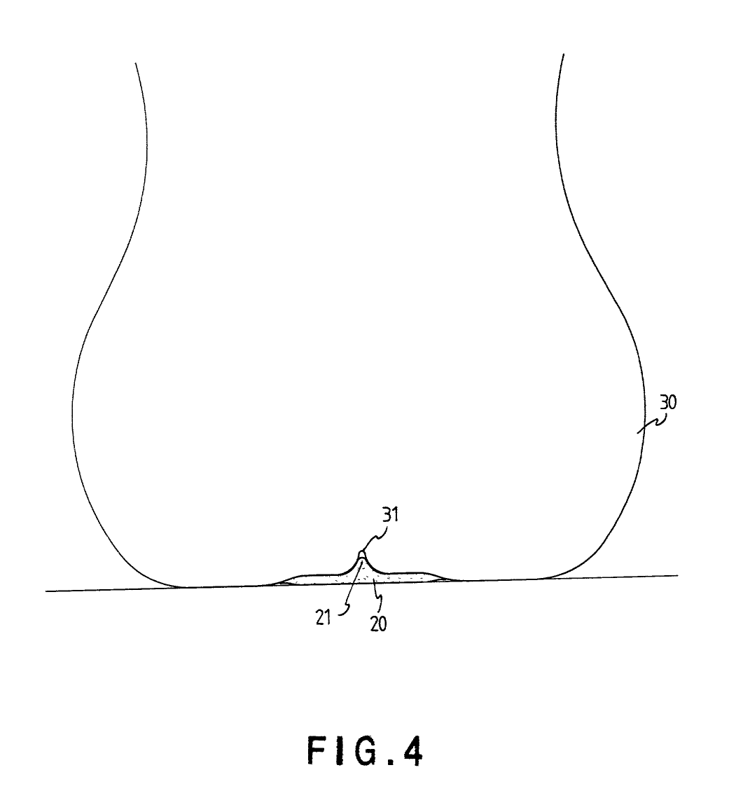

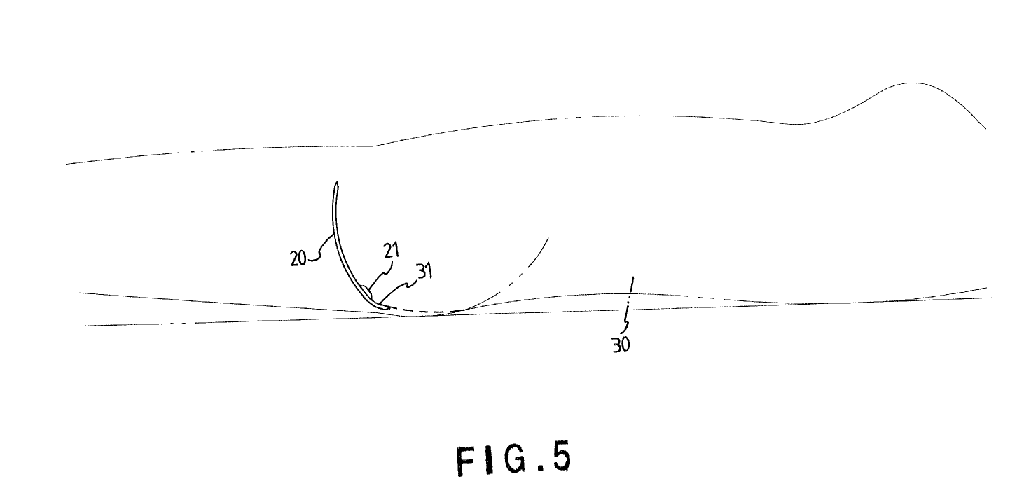

Referring to FIGS. 3 to 5, the hygienic pad of the present invention comprises a flat body 20 which is made of absorbable material and a protrusion 21 extends from a top surface of the body 20. The protrusion 21 has a tapered ridge and is shaped as a triangular prism so that it is easily inserted into the posterior groove 31 of the user and the protrusion 21 is located at a rear portion of the posterior groove and below the user's dimple.

The protrusion 21 is blocked in the rear portion of the user's posterior groove 31 and because the protrusion 21 is located at the lower position whichever status the user is in so that the menses liquid will be stopped by the protrusion 21 and absorbed by the hygienic pad.

Although the present invention has been described with reference to the preferred embodiments, it will be understood that the invention is not limited to the details described thereof. Various substitutions and modifications can be made by those skilled in the art and all such substitutions and modifications are intended to be embraced within the scope of the invention as defined in the appended claims.

What is claimed is:

1. A hygienic pad comprising:
a flat body and a protrusion extending from a top surface of said body.

2. The hygienic pad as claimed in claim 1 wherein said protrusion has a tapered ridge.
